(19) United States
(12) Patent Application Publication
(10) Pub. No.: US 2004/0210118 A1
(43) Pub. Date: Oct. 21, 2004

(54) In situ detection of endoleak and endotension

(76) Inventor: Michel Letort, Prevessins (FR)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/419,324

(22) Filed: Apr. 18, 2003

(51) Int. Cl. A61B 5/00

(52) U.S. Cl. 600/339

(57) ABSTRACT

Endotension, the continued expansion and tensioning of a blood vessel at an aneurysm site after the repair of the aneurysm by placement of a stent graft therethrough, typically caused by refilling of the aneurysmal location by blood leakage thereto, is detected. The detection is accomplished by locating a detector in the aneurysmal vessel, between the blood vessel wall and the stent graft, and monitoring signals generating therefrom which are indicative of the arrival of fresh blood in the region, and thus a leakage of blood therein. The detector may be an oxygen sensor, and the monitoring can include maintaining a history of periodic blood oxygen signals indicative of the oxygen content of blood between the stent graft and the vessel wall, and the periodic analysis of these signals to determine whether the oxygen level is increasing, indicating the existence of endoleak and thus likely endotension.

### BACKGROUND OF THE INVENTION

1. Field of the Invention

The present invention relates to the field of blood vessel repair, more particularly to the field of the detection of leakage of blood past stent grafts placed into blood vessels to enable the repair or bypass of an aneurysm in the blood vessel.

2. Description of the Related Art

Aneurysms occur in blood vessels in locations where, due to age, disease or genetic predisposition, the blood vessel strength or resiliency is insufficient to enable the blood vessel wall to retain its shape as blood flows therethrough, resulting in a ballooning or stretching of the blood vessel at the limited strength/resiliency location to form an aneurysmal sac. If the aneurysm is left untreated, the blood vessel wall may continue to expand, to the point where the remaining strength of the blood vessel wall is below that necessary to prevent rupture, and the blood vessel will fail at the aneurysm location, often with fatal result.

To prevent rupture of the aneurysm, a stent or graft of a tubular construction is introduced into the blood vessel, such as from a remote location through a catheter introduced into a major blood vessel in the leg and pushed through the blood vessel to the aneurysm location, and the stent is secured in a location within the blood vessel such that the stent spans the aneurysmal sac. The outer surface of the stent, at its opposed ends, is sealed to the interior wall of the blood vessel at a location where the blood vessel wall has not suffered a loss of strength or resiliency, such that blood flowing through the vessel is diverted through the hollow interior of the stent, and thus diverted from the blood vessel wall at the aneurysmal sac location. Therefore, the risk of rupture of the blood vessel wall at the aneurysmal location is significantly reduced, if not eliminated, and blood can continue to flow through to the downstream blood vessel without interruption.

Although the use of stent grafts to treat aneurysms is a well-developed procedure, complications can arise which, if unaddressed, can lead to failure of the aneurysm repair. In particular, there is a risk of endotension, i.e., that the aneurysmal sac will become refilled with blood, at systemic pressure. If this occurs, there is a renewed risk of blood vessel wall failure and thus serious medical complications or death. Typically, blood can enter the aneurysmal sac, after the placement of the stent graft, by virtue of an endoleak, which may include mechanism such as blood introduction by: leakage through the graft material; passage of blood between the stent graft and blood vessel wall through failure of one of the seals between the ends of the stent graft and the blood vessel wall; or, supply of blood by side branch arteries through the weakened blood vessel wall. An endoleak can be difficult to detect. Typical methods include the use of x-rays, ultrasound of CT scan technologies to monitor the status of the aneurysmal sac, and hopefully detect enlargement or refilling of the aneurysmal sac with blood before vessel wall rupture occurs. However, these techniques cannot always detect small changes in the aneurysmal sac, i.e., endotension, since the size or morphology of the aneurysmal sac may not substantially change, or may not provide a visibly detectable change, when fresh blood enters. Thus an endoleak may not be detectable when a high risk for post intervention rupture of the aneurysm exists.

Therefore, there is a need in the art to enable the detection of endoleak and endotension conditions after the placement of a stent graft to bypass the weakened blood vessel wall, with increased likelihood of early detection of a high-risk condition, without the need to resort to traditional diagnostic techniques.

### SUMMARY OF THE INVENTION

The present invention generally provides methods and apparatus for detecting endoleaks, and thus the resulting endotension, in an aneurysmal location. In one embodiment, the invention provides a detector, located within an aneurysmal blood vessel, which is responsive to the flow of fresh blood into the aneurysmal region of the blood vessel, and is configured to enable remote sensing of the detector and/or signals emitted by the detector, to evaluate the presence or absence of fresh blood within the aneurysmal blood vessel. One method for determining endoleak or endotension includes the steps of providing a detector responsive to the presence of fresh blood in the aneurysmal blood vessel area, and reading of such detector from a remote location to diagnose endoleak or endotension at the aneurysmal location.

In another embodiment, the detector is responsive to a gaseous component of blood, such as oxygen or carbon dioxide which would be present in fresh blood entering such anueral sac. In still another embodiment, the detector detects oxygen in the space between an intervention device, such as a stent graft inserted within the blood vessel to span the aneurysmal blood vessel wall area, and the wall of the aneurysmal blood vessel, and a reading and storing mechanism is provided to store a multiplicity of oxygen level readings. A remote reading device is provided to periodically review the stored readings for blood oxygen level in the space, to enable the detection of changed or increasing oxygen levels in the space between the stent graft and the aneurysmal blood vessel wall, and thereby detect the presence of endoleak or endotension. In still another embodiment, a sensor responsive to a different component of blood, such as carbon dioxide or carbon monoxide, is used in place of an oxygen sensor to detect endoleak and endotension.

In a still further embodiment, the invention provides a method of enabling detection of endoleaks or endotension without the need to resort to MRI, CT scan or ultrasound techniques, by providing an oxygen sensor implantable in the aneurysmal location, providing a time sensitive clocked reading mechanism capable of receiving signals emanating from the oxygen sensor upon placement thereof in the aneurysmal sac location, and instructions on the placement, interconnection and use of signals emanating from the oxygen sensor. In still a further embodiment, the method includes the steps of implanting the oxygen sensor in an appropriate location; interconnecting the oxygen sensor to an implantable reading and recording mechanism, and remotely sensing the reading and recording medium to receive data indicative of oxygen content at the implant location. In still another embodiment, a sensor responsive to a different component of blood, such as carbon dioxide or carbon monoxide, is used in place of an oxygen sensor to detect endoleak and endotension.

In still a further embodiment, an oxygen sensor is implantable in the aneurysmal location, providing a time sensitive clocked reading mechanism capable of receiving signals emanating from the oxygen sensor upon placement thereof in the aneurysmal location, and instructions on the placement, interconnection and use of signals emanating from the oxygen sensor. A real time monitor can be provided, which includes an alarm circuit responsive to a change in the aneurysmal blood vessel status. In still another embodiment, a sensor responsive to a different component of blood, such as carbon dioxide or carbon monoxide, is used in place of an oxygen sensor to detect endoleak and endotension.

### BRIEF DESCRIPTION OF THE DRAWINGS

FIG. 1 is a partial sectional view of an aneurysmal blood vessel, in the specific embodiment shown, an aneurysmal aorta, having a stent graft received therein and bypassing the aneurysmal portion of the vessel;

FIG. 2 is a schematic view of the placement of a sensor and controller in the chest of a patient, including a schematic placement of a reader thereby;

FIG. 3 is an enlarged view of the aneurysmal blood vessel of FIG. 1, showing a sensor received therein in the space between the stent graft and the aneurysmal blood vessel wall;

FIG. 4 is a schematic view of a controller used in the present invention; and

FIG. 5 is a schematic view of a remote alarm useful in conjunction with the detector arrangement of FIGS. 1 to 4.

### DETAILED DESCRIPTION

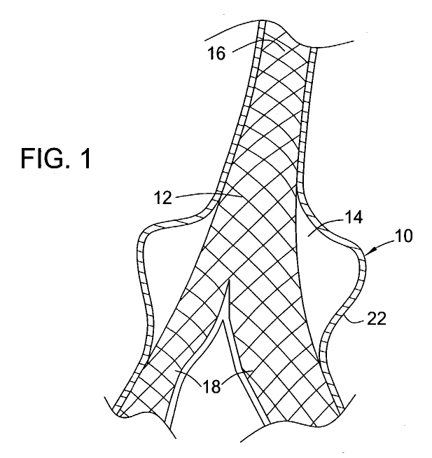

Referring initially to FIG. 1, there is shown in partial section, an aneurysmal aorta 10, having a stent graft 12 inserted therein to bypass the weakened blood vessel wall at the aneurysmal location, and thereby prevent further expansion or failure of the lumen wall at the aneurysmal location. Stent graft 12 is inserted into the aorta to span the aneurysmal sac in the aorta 10, such that the opposed ends 16, 18 of the stent graft are positioned in sealing engagement with portions of the blood vessel wall which are not implicated in the aneurysmal event, i.e., they are not in a weakened state. The placement of stent graft across the aneurysmal sac 14 causes that portion of the sac 14 to be isolated from the blood flowing through the blood vessel by the presence of stent graft 12. Thus, the stent graft provides a secure bypass of the aneurysmal blood vessel wall preventing blood flow thereto and thus eliminating systemic pressure which would otherwise result in further extension or failure thereof. The stent graft 12 is preferably introduced remotely, such as by the use of a wire and catheter inserted into a blood vessel at a remote location and then pushed therethrough to the location of the aneurysmal blood vessel, as is well known in the art. Although the stent graft is shown placed in an aorta, other aneurysmal locations, and thus other stent graft configurations and placements are specifically contemplated for use with the present invention.

After the stent graft 12 is in place, blood supply to the aneurysmal sac 14, the stent graft 12 isolates the aneurysmal sac 14 such that blood flow thereto should be substantially reduced, if not eliminated. Preferably, no fresh blood should be introduced to aneurysmal sac 14, because the presence of such blood could result in further expansion or rupture of the weakened blood vessel wall 22 in the aneurysmal sac 14. Rupture of the aneurysmal aorta 10 could lead to uncontrolled massive hemorrhaging, and patient morbidity and mortality, despite the presence of the stent graft 12 spanning the aneurysmal location.

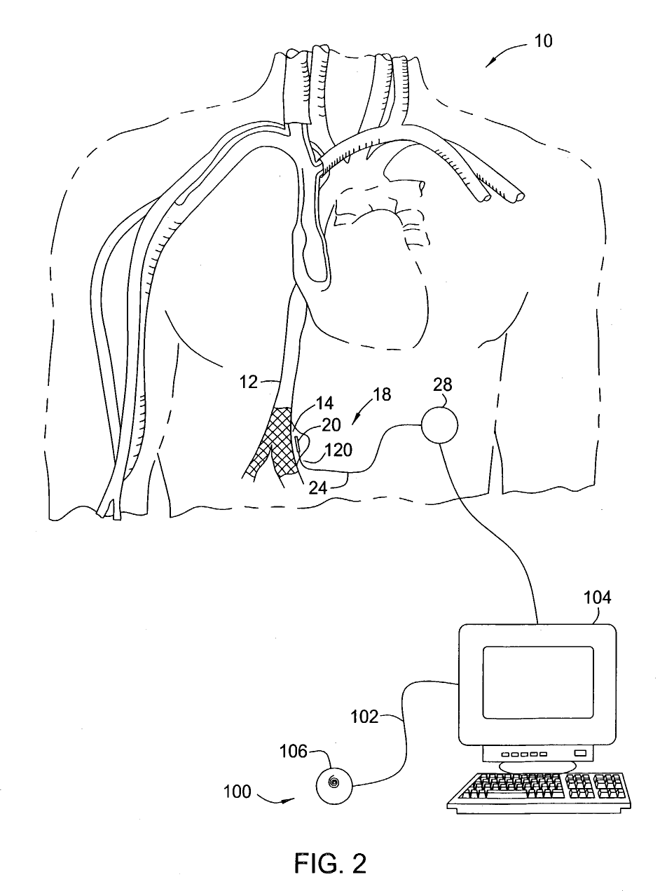

Referring now to FIG. 2, there is shown an endoleak and/or endotension detector 18 in situ, comprising an implantable oxygen sensor 20, positioned on the terminus of a lead 24 extending through the blood vessel wall 22 and terminating within the aneurysmal sac 14 between the stent graft 12 and the weakened blood vessel wall of the aneurysmal sac 14, and extending outwardly from the aneurysmal sac 14 to an implantable controller 28 preferably maintained in the abdominal cavity at a location adjacent to, but spaced from, the aneurysmal sac 14. Controller 28 is preferably configured to control the operation of oxygen sensor 20, record the readings for oxygen concentration or content generated by oxygen sensor 20, and to be remotely read, such as by a telemetry reader 100, as will be further described herein.

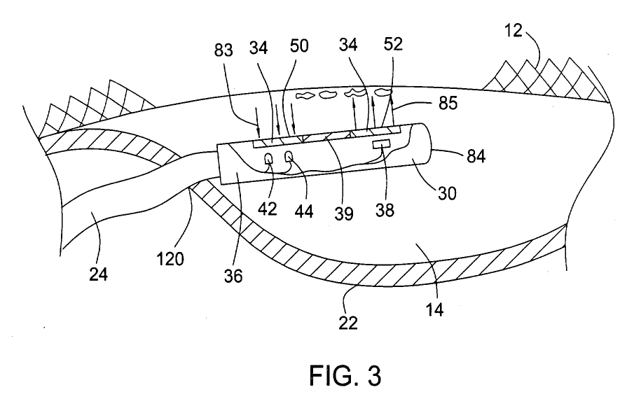

Referring now to FIG. 3, the placement of the oxygen sensor within the aneurysmal sac 14 is illustrated. The oxygen sensor 20 is shown, in partial cutaway, located in aneurysmal sac 14, and is configured to provide a signal, indicative of the level or saturation of oxygen in blood, and includes a sealed body portion 30, from which lead or leads 24 extend, a lens assembly 34 enabling light transmission inwardly and outwardly of the body portion 30 within which body portion 30 is provided a light source 36 and a light detector 38. The body portion 30 is preferably substantially miniaturized, such that its diameter is on the order of 3 to 4 mm, and the overall length thereof, non-inclusive of the lead 24, is on the order of one centimeter. A hook (not shown), or other securement mechanism, may be provided on body portion to secure the body portion 30 against withdrawal from the aneurysmal sac 14.

Oxygen sensor 20 operates on the principle that oxygen in blood reflects or absorbs light in certain frequency ranges. Thus, by emitting a known intensity of light in a specific frequency range, and detecting the intensity of light received back at the detector in that specific frequency range, the likely concentration of oxygen in the blood is ascertainable. Specifically, as shown in FIG. 3, the light source 36 preferably includes a first light emitter, preferably an LED, 42, and a second light emitter, preferably an LED, 44. First light source 42 is preferably selected to emit light having a first blood oximetry frequency, and second light source is selected to have a second blood oximetry frequency, such that by sequentially powering the light sources 42, 44 to emit light at different times, light detector 38 will detect discrete signals corresponding to different blood oximetries. The light sources 42, 44, as well as the light detector 38, are maintained within sealed body 30 adjacent lens assembly 34, which preferably includes discrete windows 50, 52, such that light sources 42, 44 are positioned adjacent window 50 and light detector 38 is positioned adjacent window 52. A non-light transmissive element 39 is positioned between the two windows 50, 52, such that light leaving the light sources 42, 44 must pass through both windows 50 and 52 before it can reach light detector 38. As light from light sources 42, 44 passes through window 50, as shown by arrows 83, it will encounter blood 84 present in the aneurysmal sac 14. A portion of such light will be directed to window 52, as shown by arrows 85, for detection by light detector 38. Further details of the operation and structure of a oxygen sensor which may be used with the present invention are found in U.S. Pat. No. 6,198,952, hereby incorporated by reference herein.

Referring again to FIG. 2, oxygen sensor 20 is connected, via lead 24, to controller 28. Lead 24 preferably has a diameter of approximately 3 to 4 mm (of the same approximate diameter as the detector body 30), and a length on the order of about 12 cm, which enables the placement of lead 24 through the aneurysmal blood vessel wall 22, such as by puncturing the wall 22 to create an opening 120 (best shown in FIG. 3) and inserting oxygen sensor 20 therethrough to position the sensor 20 in the aneurysmal sac 14 and extending the lead 24 therefrom and through the opening 120 and to the controller 28 remotely located from the aneurysmal blood vessel wall 22 location as shown in FIG. 2.

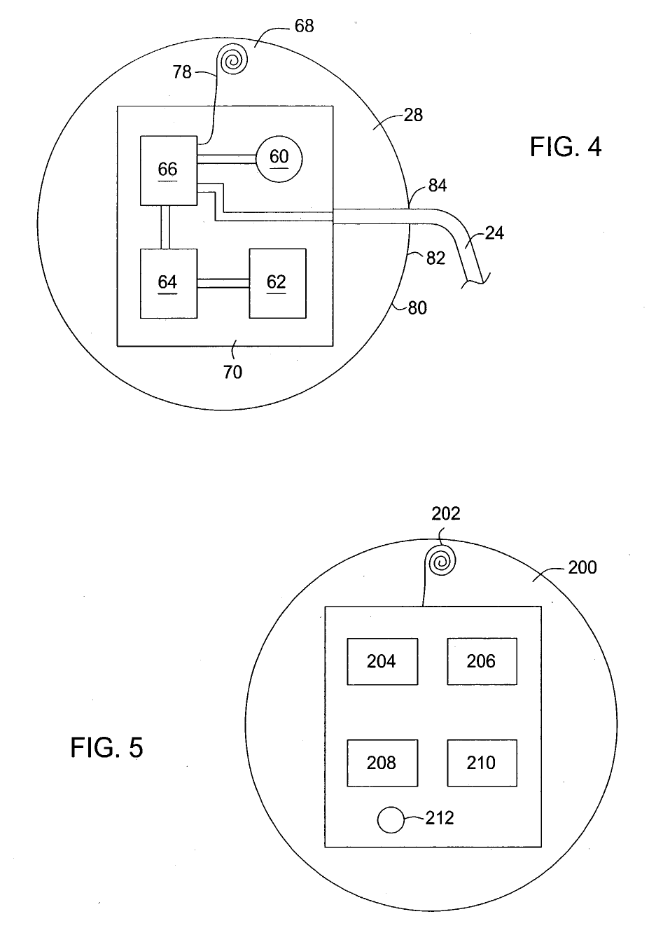

Referring now to FIG. 4, the Controller 28 is shown in a schematic form, and is provided to operate the sensor 20, and to maintain a record of the readings provided thereby of blood oxygen content for further review. The controller 28 generally includes a power supply 60, a timer/clock 62, a recording memory 64, a microprocessor 66 and a telemetry system 68 interconnected on a board 70 to enable the control of oxygen sensor 20 for detecting of oxygen content in aneurysmal sac 14, and to maintain a time based record of such measured values for later interpretation. In operation, microprocessor 66 receives a timing signal from the timer/clock 62, which is read by microprocessor 66 to establish a time gap over which to initiate a reading of oxygen content in aneurysmal sac 14. The time gap between measurements is selected to allow a maximum number of measurements to be taken and stored in memory 64 while simultaneously allowing sufficient time between remote reading of memory 64 such that memory 64 can be overwritten with new oxygen content measurements after the recorded measurements are read or otherwise used. A typical controller has sufficient memory to enable oxygen sensor 20 to make readings at 15-second intervals for 6 months, before the memory will begin re-writing over previously stored data. When microprocessor 66 has received sufficient clock signals indicative of an appropriate time interval, microprocessor 66 initiates a measure signal which opens and then closes gates or switches to pass a signal through lead 24 to sequentially power the LED's 42, 44 (FIG. 3), the period between their opening and closing being controlled such that each sequential pulse of light emitted from LED's 42, 44 is of the same time duration, typically on the order of 0.1 seconds. Opening and closing of the gates causes a connection and disconnection of light sources 42, 44 to power supply 62, such that each light source is powered to emit light in a desired frequency range for a select duration of time, such that the light from each light source 42, 44 reaches detector 38 (FIG. 3) at distinct separate periods of time. Preferably lead 24 includes at least two conductors therein, such that one conductor is connected to each LED, and the signals emitted by the light detector 38 may be returned down one or either such conductor, the light detector 38 being protected from the incoming signals from the controller 28 by circuit elements, such as a diode (not shown). Upon the sending of the signal to one or the other LED 42, 44, the microprocessor 66 also simultaneously opens gates or switches to connect detector 38 to memory 64, such that a first signal corresponding to light reaching detector 38 from first light source 42 is recorded at a first memory location and a signal corresponding to light reaching the detector 38 from the second light source 44 is recorded at a second memory location. Each recorded signal includes address information providing a time of recording linked to the recorded sensor value. Microprocessor 66 also monitors the memory 62, to detect the return of signals corresponding to the light pulses emitted from light sources 42, 44. Although the invention has been described in terms of emitting light in separate frequency ranges at separate times, the oxygen sensor could be configured to emit light in only one frequency, or, where multiple frequencies are emitted from sources 42, 44, the light detector 38 can be configured to discriminate between frequencies received, and send separate signals to the memory 62 indicative of each frequency range detected. Likewise, a plurality of light detectors, each capable of detecting light in a discrete frequency range not overlapping with the frequency range of the other may be used. Additionally, the microprocessor may be programmed to alternately power the LED's 42, 42, such that every other signal recorded will be indicative of light received at the first frequency, and thus every intervening signal recorded will be indicative of light intensity in the second frequency range

Once signals corresponding to such light pulses are received at memory 62, or, if no signal is received after a time out period corresponding to an expected return signal period is passed, the switch or gate from light detector 38 to memory 62 is closed, to prevent the recording of spurious signals in memory 62. Power supply 60 is preferably a battery selected to supply sufficient energy to power the system for a period of one to two years.

Controller 28 is preferably housed in a generally cylindrical biocompatible housing 80, having a generally thin wall 82 and configured to receive board 70, with the controller circuitry such as microprocessor 66, etc., therein. A sealed aperture 84 enables lead 24 to extend through wall 82 and thus be connected to board 70 for interconnection to the elements of controller 28. Controller 28 is preferably positioned in a sub-dermal abdominal location spaced from the aneurysm location.

To evaluate the status of the aneurysmal sac 14, a medical practitioner can read the data stored in the memory 66. Referring again to FIG. 2, a reader 100, linked via an electrically conducting cable 102 to a computer or processor 104, is positioned over the sub dermal location where controller 28 is positioned. To enable reading of memory through the skin of a patient, controller telemetry system 68 includes an antenna 78, which is linked through the board 68 and microprocessor 66, to memory 62. Likewise, reader 100 includes a read antenna 106, which is connected through cable 102, to processor 104. To initiate reading of the data stored in memory 62, the practioner first locates or creates a file in processor 104 corresponding to the downloading of the stored data. The program can be individual patient or person specific, and may include means for generating an ID signal which corresponds to a key in controller microprocessor 66 which will only allow reading of data from memory 62 upon first supplying the ID. The processor 104 sends a signal, through cable 102, to read antenna 106, which transmits a corresponding rf or other radio signal which is picked up by antenna 78 and transmitted through the interconnections on board 68 to microprocessor 66. Microprocessor 66 decodes the signal, and if appropriate, begins reading the oxygen sensor readings and transmitting them, through appropriate rf or other radio signals, through antenna 78 to read antenna 106, and thus to processor 104. An appropriate program in processor then tabulates the data, and converts the readings of the photo detector 38 into readings indicative of the oxygen content in the fluid in the aneurysmal sac 14 between the stent graft 12 and the aneurysmal wall 22 at the aneurysm 10 locations. The data can be presented in tabular full data form, graphical form, or as distinct summaries or averages of data over specified time periods, or as excursions from a tabulated norm, median or mean of all the data and corresponding times at which such excursion occurred. From the data, and from the results of the data, the practitioner evaluating the data can determine if further investigation of the conditional of the aneurysmal sac is warranted. The placement and design of controllers and readers, as well as telemetry devices capable of allowing reading of the stored memory through the skin, is well known to those skilled in the art of placement and use of implantable devices, in particular implantable devices used for the monitoring and control of the heart, such as pacemakers.

In an additional embodiment, a portable excursion monitor 200 as shown in FIG. 5 may be provided, which is configured to be carried on a patient in a location adjacent to the sub dermal location of controller 28, and is able to send and receive signals therefrom. In this embodiment, the excursion monitor 200 includes all of the interactive features of the processor 102 and reader 100, but need not include the analysis enabled by processor 104. Specifically, monitor 200 is configured to include an antenna 202, interconnected through a monitor board to a monitor microprocessor 204, a monitor memory 206 and a monitor timer/clock 208, and an alarm 210, all powered by a battery 212. In operation, microprocessor 204 periodically, at specified times set by the user or medical practitioner and set in an addressable memory 206, reads the memory 62 of readings of oxygen sensor 20 and establishes an average, mean, median or other analytical value thereof as well as a deviation for the readings. If a reading deviates from the average, mean, median or other analytical value of the just read, or previous readings of the oxygen sensor values, in a direction and quantity which indicates that fresh blood is entering the aneurysmal sac 14 existing between the stent graft 12 and the aneurysmal wall 22, the microprocessor 204 generates a signal and routes it to alarm 210, thereby activating the alarm 210 and providing the patient, or a caregiver, an early warning of the need for imminent medical attention. Alternatively, the monitor 200 circuitry could be modified, such that the controller 28 provides the analysis of the blood oxygen readings, and supplies an alarm signal though antenna 78 to antenna 202, which signal is routed to alarm 210 to activate the alarm in the event that sensor readings indicate the presence of fresh blood in the aneurysmal sac 14.

The placement of the endoleak/endotension detector 18 is accomplished surgically. Initially, a stent graft 12 is located in the blood vessel as shown in FIG. 1, in which the aneurysmal sac 14 is located, spanning the weakened aneurysmal region of the blood vessel wall 22 and having opposed ends 16, 18 in sealed contact, about the perimeter of the stent graft 12, to the internal surface of healthy blood vessel tissue. The stent graft 12 is placed by medically accepted techniques, typically through passing a catheter, with the stent graft 12 therein, in a leg blood vessel and pushing the stent graft 12 through the vessel to a desired location where it is positioned to span the aneurysmal sac 14. The placement and sealing of stent grafts 12 in place is well known to those skilled in the art.

Once the stent graft 12 is properly positioned, the extent of extension of the blood vessel at the aneurysmal site can be evaluated, and a determination made of the need to place an oxygen sensor into the aneurysmal sac 14 formed between the stent graft 12 and the blood vessel wall. Upon a determination that the oxygen sensor is appropriate, an incision is made through the patients' skin, and an endoscope (not shown) is used to direct the oxygen sensor 20 into a location adjacent the exterior of the aneurysmal blood vessel wall 22. The endoscope preferably includes a needle actuable at the distal end thereof, which needle is used to puncture the wall and thus create an aperture 120 (as shown in FIG. 3) through the blood vessel wall having a relaxed diameter slightly smaller than the diameter of lead 24. The puncture location is chosen to be a location in the blood vessel wall which is not significantly effected by the aneurysm, i.e., it has not yet suffered significant stretching or loss of resiliency. The oxygen sensor 20 is then manipulated at the end of the endoscope to be inserted through the aperture 120, such that the oxygen sensor 20 is fully received in the aneurysmal sac 14, and lead 24 extends therefrom, as shown in FIGS. 2 and 3. The endoscope is removed and the controller 28, linked to the free end of lead 24, is then located in a sub-coetaneous location as shown in FIG. 2, and sutured into place. The incision is then closed, and the patient allowed to recover. Preferably, the operation of the oxygen sensor 20 is checked immediately before implantation into the body. Over a relatively short period of time the blood vessel wall 22 will heal to seal aperture 120 about lead 24. At a later date, the resulting values for oxygen detected in the blood in the aneurysmal sac 14 are evaluated, and if an elevated oxygen content indicative of new or fresh blood entering the space is found, intervention steps may be taken to prevent further deterioration of the aneurysm. Alternatively, a sensor such as a carbon dioxide or carbon monoxide sensor can be used in place of oxygen sensor, to likewise detect changes in that gas content in the fluid in the aneurysmal sac 14 indicative of fresh blood supply thereto.

Although the invention has been described specifically in terms of the use of an oxygen sensor to evaluate the possibility of blood leakage or supply to the aneurysmal sac 14, it is specifically contemplated that other sensors, such as carbon dioxide or carbon monoxide detectors can be used to detect fresh blood incursion into the aneurysmal sac 14.

I claim:

1. An apparatus for detecting endoleak and endotension, comprising:
a detector, located within a bypassed aneurysmal blood vessel, and having a detection member capable of detecting a condition in the bypassed aneurysmal blood vessel indicative of fresh blood therein;
a controller in communication with said detector and enabled to control the operation of said detector to make detection readings; and
a reader configured to evaluate said detection readings and thereby indicate the presence of fresh blood in the bypassed aneurysmal blood vessel.

2. The apparatus of claim 1, wherein said detector is an oxygen sensor.

3. The apparatus of claim 2, wherein said oxygen sensor includes at least one light source and one light detector.

4. The apparatus of claim 1, wherein said controller includes:
a microprocessor;
a memory interconnected to said microprocessor; and
a clock.

5. The apparatus of claim 4, wherein said microprocessor controls the operation of said detector, and actuates said detector to detect conditions indicative of fresh blood present in the bypassed aneurysmal blood vessel and to transmit a signal indicative of the condition detected.

6. The apparatus of claim 5, wherein said microprocessor further directs said signal to said memory.

7. The apparatus of claim 6, wherein said signals are recorded in said memory in conjunction with an indication of the time said signal was generated.

8. The apparatus of claim 7, wherein said detector includes at least a first and a second light source, and at least one light detector.

9. The apparatus of claim 8, wherein said light sources emit radiation at different frequencies.

10. The apparatus of claim 9, wherein said microprocessor is configured to cause each one of said light sources to emit radiation in a separate time window.

11. The apparatus of claim 10, wherein said light sources emit light simultaneously and said light detector transmits a signal indicative of the intensity of light reaching the light detector at each frequency to said memory.

12. A method of providing detection of aneurysm repair irregularity following aneurysm repair, comprising the steps of:
providing a sensor indicative of the presence of at least one fluid constituent entering the space between an aneurysm repair vehicle and the aneurysmal wall;
providing a mechanism to operate and interpret any data generated by the detector; and
instructing the placement of the sensor, at a location adjacent the aneurysmal wall.

13. The method of claim 12, wherein the instructed location is within the space existing between the aneurysm repair vehicle and the aneurysmal wall.

14. The method of claim 13, wherein the sensor is an oxygen sensor.

15. The method of claim 14, wherein the oxygen sensor includes at least one light sensor therein.

16. The method of claim 15, wherein the step of providing a mechanism to operate and interpret any data generated by the detector further includes the steps of;
providing a controller capable of;
initiating, with said controller, the detection of a property of a fluid constituent located in the space between the aneurysm repair vehicle and the aneurysmal wall with the sensor;
receiving from the sensor, with the controller, a signal indicative of a property of the fluid; and
storing, in a memory associated with the controller, a signal corresponding to the property of the fluid along with a corresponding marker related to a time when the signal indicative of the fluid property was generated.

17. The method of claim 16, further including the steps of enabling the controller to initiate a multiplicity of initiations of signals from the sensor indicative of the properties of a fluid located in the space between the aneurysm repair vehicle and the aneurysmal wall, and to store such signals each with a corresponding marker indicative of the time when such signal indicative of the fluid property was generated.

18. The method of claim 12, wherein the step of providing a mechanism to operate and interpret any data generated by the detector; further includes the steps of:
providing a controller capable of;
initiating, with said controller, the detection of a property of a fluid constituent located in the space between the aneurysm repair vehicle and the aneurysmal wall with the sensor;
receiving from the sensor, with the controller, a signal indicative of a property of the fluid;
storing, in a memory associated with the controller, a signal corresponding to the property of the fluid along with a corresponding marker related to a time when the signal indicative of the fluid property was generated;
providing a telemetry member coupled to the controller; and
providing a reader, remote from the controller, capable of sending signals to the controller, through the telemetry system, to initiate the return of signals, from the controller, indicative of signals recorded in memory including the accompanying time marker therefore.

19. The method of claim 18, further including the steps of detecting a biochemical property of the fluid with the sensor.

20. A method of detecting endotension in a blood vessel following aneurysm repair, comprising the steps of:
providing a sensor connected by a lead to a controller;
positioning said sensor into the space between an aneurysm repair vehicle and the wall of the blood vessel having an aneurysmal event;
directing the sensor, with the controller, to initiate a sensing of a property of a fluid present in the space between an aneurysm repair vehicle and the wall of the blood vessel having an aneurysmal event;
reading, by the sensor, at least one biochemical property of a fluid in the space between the aneurysm repair vehicle and the wall of the blood vessel having an aneurysmal event;
generating a signal from the sensor indicative of the property and transmitting the signal from the sensor to the controller; and
recording the signal in a memory in the controller.

21. The method of claim 20, further including the steps of:
directing, with the controller, a plurality of discrete sensings of at least one property of a fluid in the space between an aneurysm repair vehicle and the wall of the blood vessel having an aneurysmal event;
reading, by the sensor, of each of the plurality of sensing;
generating, by the sensor, a signal indicative of the a the property for each reading by the sensor and transmitting each signal to the controller;
recording each signal in a memory associated with the controller; and
generating a marker, indicative of the time each signal was generated and associating such markers with corresponding signals received in the memory.

22. The method of claim 21, further including the step of measuring, by the readings of the sensor, the oxygen content of the fluid in the space between an aneurysm repair vehicle and the wall of the blood vessel having an aneurysmal event.

23. The method of claim 22, wherein the oxygen content of the fluid is measured by:
directing, into the fluid, light at a selected intensity in a frequency known to be absorbed or otherwise effected by oxygen in blood;
detecting the intensity of light passing through the fluid; and
generating a signal indicative of the intensity of light so detected.

24. (currently amended): The method of claim 23, further including the step of transmitting the signal indicative of the intensity of light detected to the memory; and
recording the signal.

25. The method of claim 23, further including the step of comparing the signal value to the intensity of light directed into the fluid.

26. The method of claim 23, further including the step of comparing the signal to signals previously generated by the sensor.

27. The method of claim 24, further including the steps of:
providing a reader, remote from the controller;
generating a signal from the reader, receivable from the controller, to initiate the controller to send data from the memory indicative of the readings received in the memory indicative of oxygen level in the fluid, coupled with markers indicative of the time when each signal corresponding to such reading was made;
transmitting the data to the reader;
transmitting the data from the reader to a processor; and
interpreting the data to provide information indicative of the oxygen content in the space between an aneurysm repair vehicle and the wall of the blood vessel having an aneurysmal event.

28. The method of claim 24, further including the steps of:
providing a monitor in a location remote from the controller;
providing a signal reception member and an alarm in the monitor;
periodically sending signals indicative of the oxygen level in the space between an aneurysm repair vehicle and the wall of the blood vessel having an aneurysmal event to the monitor signal reception member;
evaluating the signals sent to the monitor; and
actuating the alarm if the oxygen content exceeds a desired level.

29. The method of claim 28, wherein the monitor includes a microprocessor and a memory;
the microprocessor periodically reads the signals received in memory and performs a function to determine whether to actuate the alarm; and
the microprocessor sends an alarm to the signal if the data is indicative of an oxygen level which is undesirable.
